(19) United States
(12) Patent Application Publication
(10) Pub. No.: US 2010/0240772 A1
(43) Pub. Date: Sep. 23, 2010

(54) PIGMENT-FREE, AQUEOUS POLYMER DISPERSIONS MARKED WITH FLUORESCENT DYES, PROCESS FOR THEIR PREPARATION AND THEIR USE

(75) Inventors: Tobias Joachim Koplin, Ludwigshafen (DE); Oliver Reimers, Weinheim (DE); Simon Nord, Karlsruhe (DE); Dirk Lawrenz, Hassloch (DE); Oliver Birkert, Fussgoennheim (DE); Ulrike Geissler, Haar (DE)

Correspondence Address: OBLON, SPIVAK, MCCLELLAND MAIER & NEUSTADT, L.L.P., 1940 DUKE STREET, ALEXANDRIA, VA, 22314, US

(73) Assignee: BASF SE, Ludwigshafen (DE)

(21) Appl. No.: 12/682,467

(22) PCT Filed: Sep. 11, 2008
(86) PCT No.: PCT/EP2008/062068

§ 371 Date: Apr. 9, 2010

(30) Foreign Application Priority Data
Oct. 9, 2007 (EP) 07118116.8

(51) Int. Cl. A01N 25/04; C08L 101/00; C08L 53/00; A61K 47/30; A61K 8/04; B01J 31/06

(52) U.S. Cl. 514/772.3; 524/500; 524/505; 524/502; 502/159

(57) ABSTRACT

Pigment-free, aqueous polymer dispersion marked with fluorescent dyes, having a viscosity of from 500 to 900 mPa·s, determined with the aid of the Brookfield method (spindle 3, 23° C.), and comprising, based in each case on the solids of the polymer dispersion,
(A) from 1 to 99% by weight of at least one type of water-soluble and/or water-dispersible polymer,
(B) from 1 to 20% by weight of at least one type of water-dispersible polymer particles which comprise at least one fluorescent dye and
(C) from 0.001 to 3% by weight of at least one thickener;
   process for its preparation and its use.

### FIELD OF THE INVENTION

The present invention relates to novel, pigment-free, aqueous polymer dispersions marked with fluorescent dyes. The present invention also relates to a novel process for the preparation of pigment-free, aqueous polymer dispersions marked with fluorescent dyes. Not least, the present invention relates to the use of the novel, pigment-free, aqueous polymer dispersions marked with fluorescent dyes and of the aqueous polymer dispersions marked with fluorescent dyes and prepared by the novel process.

### PRIOR ART

Aqueous dispersions which comprise water-dispersible polymer particles which have a volume-average particle size of from 1 to 100 μm, determined by light scattering, and comprise a fluorescent dye are disclosed in European patent application EP 1 801 127 A1. They are known to be obtainable by free radical suspension polymerization of ethylenically unsaturated monomers in an oil-in-water emulsion whose disperse phase comprises at least one fluorescent dye in solution. Preferably, the suspension polymerization is carried out in the presence of protective colloids, such as cellulose derivatives or polyvinyl alcohols.

It is known that the protective colloids are absorbed in loops on the particle surface of the relevant polymer particles (cf. Römpp Online 2007, “Schutzkolloid”). In an aqueous dispersion which additionally comprises other polymers, in particular polymer particles, the protective colloids therefore generally have no thickening effect.

EP 1 801 127 A1 also discloses paper coating slips which have a viscosity of 1000 mPa·s, the manner in which the viscosity has been measured not being stated. The paper coating slips comprise large amounts of inorganic pigments and small amounts of a styrene/butadiene binder and of a thickener and small amounts of polymer particles which comprise at least one fluorescent dye. The paper coating slips are prepared by mixing their constituents with one another in a stirred vessel. Whether pigment-free, aqueous dispersions which already have styrene/butadiene binders and polymer particles comprising at least one fluorescent dye and a thickener are prepared separately beforehand for this purpose is not known. The properties which these material subcombinations might have considered by themselves are therefore not evident from EP 1 801 127 A1.

EP 1 801 127 A1 furthermore discloses a pigment-free paper size which is obtained by mixing aqueous dispersions comprising polymer particles which comprise at least one fluorescent dye with a standard dispersion of acrylate copolymers and stirring the resulting mixture into an aqueous starch solution, the ratio of synthetic product to starch being about 1:100. The starch is therefore present in a very large excess and not in a concentration as usually used in the case of thickeners. Moreover, the ratio of polymer particles which comprise at least one fluorescent dye to the polymer particles of the standard dispersion is not stated.

In the preparation of functional products, such as, for example, coating materials, adhesives, sealing compounds, printing inks, paper coating slips or paper sizes which are marked with at least one fluorescent dye, it would be a major advantage to have in stock pigment-free, aqueous polymer dispersions which are marked with fluorescent dyes and have water-soluble and/or water-dispersible polymers and water-dispersible polymer particles comprising at least one fluorescent dye, in order to be able to use them as required also at different locations for different intended uses and/or at different times. As a result, in the preparation of the relevant functional products, it would be possible to dispense with at least one mixing step on site, which would be a major economic and technical advantage.

However, this requires that the relevant pigment-free aqueous polymer dispersions marked with fluorescent dyes have a high long-term stability and show no settling of the polymer particles in particular for at least 1.5 months on storage at rest. Although this could also be achieved by the addition of thickeners, a simple increase of the viscosity does not offer a satisfactory solution because the relevant, pigment-free, aqueous polymer dispersions marked with fluorescent dyes must still remain pumpable so that they can be handled without excessive energy consumption in the plants for the preparation of the abovementioned functional products. In addition, the relevant pigment-free, aqueous polymer dispersions marked with fluorescent dyes must also remain stable and must not flocculate during the shearing which inevitably takes place during pumping.

However, EP 1 801 127 A1 gives no suggestions and indications as to how these problems could be solved.

### OBJECT

It was therefore the object of the present invention to provide a novel, pigment-free, aqueous polymer dispersion marked with fluorescent dyes, which polymer dispersion comprises
(A) at least one type of water-soluble and/or water-dispersible polymer and
(B) at least one type of water-dispersible polymer particles which comprise at least one fluorescent dye,
   and which has a high long-term stability in combination with outstanding pumpability. In particular, the novel, pigment-free, aqueous polymer dispersion marked with fluorescent dyes should show no settling on storage at rest for at least 1.5 months. Moreover, it should be capable of being handled, in particular pumped, without a high energy consumption and without flocculation occurring during shearing. It should thus become possible to stock the novel, pigment-free, aqueous polymer dispersion marked with fluorescent dyes, in order to be able to use it as required also at different locations for different intended uses and/or at different times. It should thus become possible to dispense with at least one mixing step on site during the preparation of functional products, such as coating materials, adhesives, sealing compounds, printing inks, paper coating slips or paper sizes, which is a major economic and technical advantage. Not least, the novel, pigment-free, aqueous polymer dispersion marked with fluorescent dyes should be capable of being mixed in a simple manner with effect-imparting additives, in particular with effect-imparting pigments, without flocculation of constituents occurring.

It was also the object of the present invention to provide a novel process for the preparation of an effect-imparting, aqueous polymer dispersion which is marked with fluorescent dyes and comprises
(A) at least one type of water-soluble and/or water-dispersible polymer,
(B) at least one type of water-dispersible polymer particles which comprise at least one fluorescent dye and
(C) at least one effect-imparting additive.

The novel process should be capable of being carried out with comparatively little energy consumption and complexity in terms of process engineering, in particular with fewer mixing steps. In addition, the novel process should make it possible to prepare the pigment-containing, aqueous polymer dispersion marked with fluorescent dyes also at different locations, for different intended uses and/or at different times, as required.

### ACHIEVEMENT ACCORDING TO THE INVENTION

Accordingly, the novel, pigment-free, aqueous polymer dispersion marked with fluorescent dyes, having a viscosity of from 500 to 900 mPa·s, determined with the aid of the Brookfield method (spindle 3, 23° C.), and comprising, based in each case on the solids of the polymer dispersion,
(A) from 1 to 99% by weight of at least one type of water-soluble and/or water-dispersible polymer,
(B) from 1 to 20% by weight of at least one type of water-dispersible polymer particles which comprise at least one fluorescent dye and
(C) from 0.001 to 3% by weight of at least one thickener
   was found, which is referred to below as “polymer dispersion according to the invention”.

The novel process for the preparation of a polymer dispersion according to the invention, in which, based on the solids of the polymer dispersion,
(A) from 1 to 99% by weight of at least one type of water-soluble and/or water-dispersible polymer,
(B) from 1 to 20% by weight of at least one type of water-dispersible polymer particles which comprise at least one fluorescent dye and
(C) from 0.001 to 3% by weight of at least one thickener and
(D) if appropriate, at least one additive stabilizing the polymer dispersion
   are homogeneously mixed in water and which is referred to below as “preparation process I according to the invention” was also found.

Furthermore, the novel use of the polymer dispersion according to the invention and of the polymer dispersion according to the invention, prepared by the process according to the invention, as marked, liquid functional products and for the production of marked, liquid, solid pulverulent and solid compact functional products which comprise at least one effect-imparting additive (E) differing from the stabilizing additives (D) was found, which is referred to below as “use according to the invention”.

Not least, the novel process for the production of marked, liquid, solid pulverulent and solid compact functional products which comprise at least one effect-imparting additive (E), in which a polymer dispersion according to the invention or a polymer dispersion according to the invention, prepared by the preparation process I according to the invention, is homogeneously mixed with at least one effect-imparting additive (E), resulting in a marked, liquid functional product or intermediate comprising at least one effect-imparting additive (E), was found.

The novel process for the production of marked, liquid, solid pulverulent and solid compact functional products which comprise at least one effect-imparting additive (E) is referred to below as “preparation process II according to the invention”.

### ADVANTAGES OF THE INVENTION

In view of the prior art, it was surprising and not foreseeable for the person skilled in the art that the object of the present invention could be achieved with the aid of the polymer dispersion according to the invention, of the preparation process I according to the invention, of the use according to the invention and of the preparation process II according to the invention.

In particular, it was surprising that the polymer dispersion according to the invention had particularly high long-term stability in combination with outstanding pumpability. The polymer dispersion according to the invention showed no settling on storage at rest for at least 1.5 months, preferably for at least 6 months and in particular for 12 months. It could be circulated by pumping, preferably by means of gear pumps, in particular in circular pipelines of production plants, without high energy consumption and without flocculation during the resulting shearing.

The polymer dispersion according to the invention could be particularly advantageously prepared in a simple manner with the aid of the preparation process I according to the invention. There, the reproducibility of a given polymer dispersion according to the invention on carrying out the preparation process I according to the invention several times was outstanding. This made it possible to prepare the polymer dispersion according to the invention in large amounts.

Thus, the polymer dispersion according to the invention, in particular that prepared by the process according to the invention, could be stocked in large amounts and it could also be used according to needs at different locations for different intended uses, but in particular for the use according to the invention, and/or at different times.

In the use according to the invention, the outstanding usability of the polymer dispersions according to the invention was evident.

Thus, they could advantageously be used as marked, liquid functional products, such as marking materials, coating materials, adhesives, sealing compounds, printing inks, paper coating slips, paper sizes and starting materials for the production of shaped articles and films.

In addition, they could advantageously be used for the production of marked, liquid, solid pulverulent and solid compact functional products which comprise at least one effect-imparting additive (E), such as marking materials, coating materials, adhesives, sealing compounds, printing inks, paper coating slips, paper sizes, starting materials for the production of shaped articles and films, catalysts, formulations of crop protection agents and cosmetic and pharmaceutical formulations.

It proved to be a very particular advantage that the functional products which comprise at least one effect-imparting additive (E) could be produced in the desired amounts from the polymer dispersions according to the invention by the preparation process II according to the invention according to needs at the respective desired times at the desired locations where suitable plants were available. It proved to be an additional particular advantage that fewer mixing steps were necessary in the preparation process II according to the invention than in known preparation processes.

Owing to their content of fluorescent dyes, the authenticity of the polymer dispersions according to the invention and of the functional products produced therefrom and of the subsequent products thereof, such as, for example, marked fuels, coatings, adhesive layers, seals, printed substrates, converted paper products, catalysts, crop protection agents, cosmetics and drugs, could be rapidly checked in a simple manner.

### DETAILED DESCRIPTION OF THE INVENTION

The polymer dispersion according to the invention is marked with fluorescent dyes. “Marked” means that the fluorescent dyes are permanently associated with the solid of the polymer dispersion and are preferably present in a concentration such that they are recognizable with the naked eye or can be detected with the aid of suitable spectroscopic methods, such as, for example, fluorescence spectroscopy. Examples of suitable fluorescent dyes for use in the polymer dispersion according to the invention are described in detail below.

Here and below, solid is to be understood as meaning the sum of all constituents of the polymer dispersion according to the invention which, after complete removal of volatile constituents, such as, for example, water or organic solvents, remain behind as residue, which is preferably solid and is not volatile in the temperature range from room temperature to preferably 250° C.

The polymer dispersion according to the invention is pigment-free. This means that it comprises at most traces of customary and known pigments, fillers and nanoparticles which do not influence the physical and chemical properties of the polymer dispersion according to the invention or do so only to an insignificant extent.

The polymer dispersion according to the invention is aqueous. In the context of the present invention, this means that water or a liquid aqueous medium which substantially comprises water forms the continuous phase of the dispersion. The term “substantially” indicates that the chemical and physical properties of the liquid aqueous medium are determined mainly by water.

The polymer dispersion according to the invention has a viscosity of from 500 to 900 mPa·s, in particular from 550 to 700 mPa·s, measured using the Brookfield method with spindle No. 3, at 23° C.

The speed of rotation of the spindle during the measurement of the Brookfield viscosity may vary and depends on the expected viscosity, so that the person skilled in the art can establish the viscosity suitable in each case on the basis of its general technical knowledge. In the case of the viscosity range according to the invention, the measurement is preferably carried out at 100 rpm.

The polymer dispersion according to the invention comprises, based in each case on its solids, from 1 to 99% by weight, preferably from 20 to 98% by weight and in particular from 30 to 97% by weight of at least one, in particular one, type of water-soluble and/or water-dispersible polymer (A). In particular, water-dispersible polymers (A) which are present in the polymer dispersion according to the invention in the form of dispersed polymer particles (A) are used.

The particle size of the polymer particles (A) may vary widely and can therefore be optimally adapted to the requirements of the individual case. Preferably, the particle sizes of the polymer particles (A) are smaller than the particle sizes of the polymer particles (B). In particular, the polymer particles (A) have a volume-average particle size of from 0.01 to 1 μm, determined by light scattering.

Suitable methods for determining the volume-average particle size with the aid of light scattering are disclosed, for example, in European patent application EP 1 801 127 A1, page 9, paragraph [0052], and page 11, paragraph [0064].

Suitable polymers (A) are in principle all polymers which are soluble or dispersible, in particular dispersible, in water without decomposing thereby and/or interacting in an undesired manner with the other constituents of the polymer dispersion according to the invention. This means that, for example, they do not undergo any chemical reaction step or form flocs therewith.

The polymers (A) are preferably selected from the group consisting of random, alternating and block, linear, branched and comb-like (co)polymers of ethylenically unsaturated monomers, polyaddition resins and polycondensation resins which, if appropriate, are crosslinkable three-dimensionally by physical drying, thermal curing and/or curing with actinic radiation.

Regarding the terms (co)polymers, polyaddition resins and polycondensation resins, reference is made to Römpp Lexikon Lacke and Druckfarben, Georg Thieme Verlag, Stuttgart, New York, 1998, page 457, “Polyaddition” and “Polyadditionsharze (Polyaddukte)”, and pages 463 and 464, “Polykondensate”, “Polykondensation” and “Polykondensationsharze”, and pages 73 and 74, “Bindemittel”.

In the context of the present invention, the term “physical drying” means the curing of a layer which comprises at least one polymer (A) and at least one solvent by film formation by removal of solvent from the layer, the linkage within the layer taking place via loop formation of the polymer molecules (A). Alternatively, the film formation takes place via the coalescence of the polymer particles (A) (cf. Römpp Lexikon Lacke and Druckfarben, Georg Thieme Verlag, Stuttgart, New York, 1998, “Härtung”, pages 274 and 275). Usually, no crosslinking agents are required for this purpose.

The thermal curing is generally effected by the thermally initiated and maintained reaction of complementary reactive functional groups, all of which may be present in the polymer (A); in such a case, a self-crosslinking polymer (A) is referred to. However, a type of reactive functional groups may be present in the polymer (A) and the complementary reactive functional groups in a heat-curable crosslinking agent (E); in this case, an externally crosslinking polymer (A) is referred to.

Examples of suitable complementary reactive functional groups are listed in the following overview. In the overview, the variable R is an acyclic or cyclic aliphatic radical, an aromatic radical and/or an aromatic-aliphatic (araliphatic) radical; the variables R¹ and R² are identical or different aliphatic radicals or are linked to one another to form an aliphatic or heteroaliphatic ring.

### Overview: Examples of Complementary Functional Groups

Binder and Crosslinking agent
Or
Crosslinking agent and Binder

| | |
|---|---|
| —SH | —C(O)—OH |
| —NH₂ | —C(O)—O—C(O)— |
| —OH | —NCO |
| —O—(CO)—NH—(CO)—NH₂ | —NH—C(O)—OR |
| —O—(CO)—NH₂ | —CH₂—OH |
| >NH | —CH₂—O—R |
| | —NH—CH₂—O—R |
| | —NH—CH₂—OH |
| | —N(—CH₂—O—R)₂ |
| | —NH—C(O)—CH(—C(O)OR)₂ |
| | —NH—C(O)—CH(—C(O)OR)(—C(O)—R) |
| | —NH—C(O)—NR¹R² |
| | >Si(OR)₂ |
| | O |
| | —CH—CH₂ |
| | O |
| | C |
| | O O |
| | —CH—CH₂ |
| —C(O)—OH | O |
| | —CH—CH₂ |
| | —N═C═N— |
| | —C(O)—N(CH₂—CH₂—OH)₂ |

The choice of the respective complementary groups depends firstly on the fact that they are not permitted to undergo any undesired reactions, in particular any premature crosslinking, during the preparation, the storage, the further processing and the use of the polymer dispersion according to the invention and/or, if appropriate, are not permitted to disturb or inhibit the curing with actinic radiation, and secondly on the temperature range in which the crosslinking is to take place.

In the case of externally crosslinking polymers (A), preferably thio, hydroxyl, N-methylolamino, N-alkoxy-methylamino, imino, carbamate, allophanate and/or carboxyl groups, preferably hydroxyl or carboxyl groups, are used on the one hand and preferably crosslinking agents having anhydride, carboxyl, epoxy, blocked isocyanate, urethane, methylol, methylol ether, siloxane, carbonate, amino, hydroxyl and/or beta-hydroxyalkylamido groups, preferably epoxy, beta-hydroxyalkylamido, blocked isocyanate, urethane or alkoxymethylamino groups, are used on the other hand.

In the case of self-crosslinking polymers (A), in particular methylol, methylol ether and/or N-alkoxy-methylamino groups are used.

In the context of the present invention, “actinic radiation” is to be understood as meaning electromagnetic radiation, such as near-infrared (NIR), visible light, UV radiation, X-rays and gamma radiation, in particular UV radiation, or corpuscular radiation, such as electron radiation, beta radiation, alpha radiation, neutron radiation or proton radiation, in particular electron radiation.

The curing with actinic radiation is effected in general via bonds which can be activated by means of actinic radiation and are present in the polymers (A). Examples of suitable bonds which can be activated by means of actinic radiation are carbon-hydrogen single bonds or carbon-carbon, carbon-oxygen, carbon-nitrogen, carbon-phosphorus or carbon-silicon single bonds or double bonds. Among these, the double bonds, in particular the carbon-carbon double bonds (referred to below as “double bonds”) are preferably used.

Suitable double bonds are present, for example, in (meth)acrylate, ethacrylate, crotonate, cinnamate, vinyl ether, vinyl ester, ethenylarylene, dicyclo-pentadienyl, norbornenyl, isoprenyl, isopropenyl, allyl or butenyl groups; ethenylarylene, dicyclopentadienyl, norbornenyl, isoprenyl, isopropenyl, allyl or butenyl ether groups or ethenylarylene, dicyclopentadienyl, norbornenyl, isoprenyl, isopropenyl, allyl or butenyl ester groups. Among these, (meth)acrylate groups, in particular acrylate groups, are particularly advantageous and are therefore very particularly preferably used.

Examples of suitable (co)polymers (A) are (meth)acrylate (co)polymers, butadiene (co)polymers or partly hydrolyzed polyvinyl esters, in particular (meth)acrylate copolymers and butadiene copolymers.

Examples of suitable polyaddition resins and/or polycondensation resins (A) are polyesters, alkyds, polyurethanes, polylactones, polycarbonates, polyethers, epoxy resin/amine adducts, polyureas, polyamides, polyimides, polyester/polyurethanes, polyether/polyurethanes or polyester/polyether/poly-urethanes, in particular polyesters.

Among these polymers (A), the (meth)acrylate copolymers and butadiene copolymers (A) have particular advantages and are therefore particularly preferably used.

Examples of suitable ethylenically unsaturated monomers (a) for the preparation of the (meth)acrylate copolymers and butadiene copolymers (A) are
(a1) monomers which carry at least one hydroxyl, amino, alkoxymethylamino, carbamate, allophanate, isocyanate, epoxide or imino group per molecule, such as
(a11) hydroxyalkyl esters of acrylic acid, methacrylic acid or another alpha,beta-olefinically unsaturated carboxylic acid which are derived from an alkylene glycol which is esterified with the acid, or which are obtainable by reacting the alpha,beta-olefinically unsaturated carboxylic acid with an alkylene oxide, such as ethylene oxide or propylene oxide, in particular hydroxyalkyl esters of acrylic acid, methacrylic acid, ethacrylic acid, crotonic acid, maleic acid, fumaric acid or itaconic acid, in which the hydroxyalkyl group comprises up to 20 carbon atoms, such as 2-hydroxyethyl, 2-hydroxypropyl, 3-hydroxypropyl, 3-hydroxybutyl, 4-hydroxybutyl acrylate, methacrylate, ethacrylate, crotonate, maleate, fumarate or itaconate; or hydroxy-cycloalkyl esters, such as 1,4-bis(hydroxy-methyl)cyclohexane, octahydro-4,7-methano-1H-indenedimethanol or methylpropanediol mono-acrylate, monomethacrylate, monoethacrylate, monocrotonate, monomaleate, monofumarate or monoitaconate; reaction products of cyclic esters, such as, for example, epsilon-caprolactone and these hydroxyalkyl or hydroxy-cycloalkyl esters;
(a12) ethylenically unsaturated alcohols, such as allyl alcohol;
(a13) polyols, such as trimethylolpropane mono- or diallyl ether or pentaerythrityl mono-, di- or triallyl ether;
(a14) reaction products of acrylic acid and/or methacrylic acid with the glycidyl ester of a monocarboxylic acid branched in the alpha-position and having 5 to 18 carbon atoms per molecule, in particular of a Versatic® acid, or, instead of the reaction product, an equivalent amount of acrylic and/or methacrylic acid, which is then reacted during or after the polymerization reaction with the glycidyl ester of a monocarboxylic acid branched in the alpha-position and having 5 to 18 carbon atoms per molecule, in particular of a Versatic® acid;
(a15) aminoethyl acrylate, aminoethyl methacrylate, allylamine or N-methyliminoethyl acrylate;
(a16) N,N-di(methoxymethyl)aminoethyl acrylate or methacrylate or N,N-di(butoxymethyl)aminopropyl acrylate or methacrylate;
(a17) (meth)acrylamides, such as (meth)acrylamide, N-methyl-, N-methylol-, N,N-dimethylol-, N-methoxymethyl-, N,N-di(methoxymethyl)-, N-ethoxymethyl- and/or N,N-di(ethoxy-ethyl)(meth)acrylamide;
(a18) acryloyloxy- or methacryloyloxyethyl, -propyl or -butyl carbamate or allophanate; further examples of suitable monomers which comprise carbamate groups are described in the U.S. Pat. Nos. 3,479,328, 3,674,838, 4,126,747, 4,279,833 or 4,340,497; and/or
(a19) acryloyloxysilane-comprising vinyl monomers which can be prepared by reaction of hydroxy-functional silanes with epichlorohydrin and subsequent reaction of the reaction product with (meth)acrylic acid and/or hydroxyalkyl and/or hydroxycycloalkyl esters of (meth)acrylic acid and/or further monomers (a1) containing hydroxyl groups.
(a110) monomers comprising epoxide groups, such as the glycidyl ester of acrylic acid, methacrylic acid, ethacrylic acid, crotonic acid, maleic acid, fumaric acid or itaconic acid or allyl glycidyl ether.
(a2) Monomers which carry at least one acid group per molecule, such as
(a21) acrylic acid, beta-carboxyethyl acrylate, methacrylic acid, ethacrylic acid, crotonic acid, maleic acid, fumaric acid, aconitic acid or itaconic acid;
(a22) ethylenically unsaturated sulfonic or phosphonic acids or the partial esters thereof, such as, for example, vinylsulfonic acid or vinylphosphonic acid;
(a23) mono(meth)acryloyloxyethyl maleate, mono(meth)-acryloyloxyethyl succinate or mono(meth)acryl-oyloxyethyl phthalate; or
(a24) vinylbenzoic acid (all isomers), alpha-methyl-vinylbenzoic acid (all isomers) or vinyl-benzenesulfonic acid (all isomers); and
(a3) monomers which are substantially or completely free of reactive functional groups, such as
(a31) (meth)acrylates substantially free of acid groups, such as alkyl or cycloalkyl (meth)acrylates having up to 20 carbon atoms in the alkyl radical, in particular methyl, ethyl, n-propyl, n-butyl, sec-butyl, tert-butyl, hexyl, ethylhexyl, stearyl and lauryl acrylate or methacrylate; cycloaliphatic (meth)acrylates, in particular cyclohexyl, isobornyl, dicyclopentadienyl, octahydro-4,7-methano-1H-indenemethanol or tert-butylcyclohexyl (meth)acrylate; oxaalkyl or oxacycloalkyl (meth)acrylates, such as ethoxytriglycol (meth)acrylate and methoxyoligoglycol (meth)acrylate having a molecular weight Mn of, preferably, 550 or other ethoxylated and/or propoxylated (meth)acrylic acid derivatives free of hydroxyl groups (further examples of suitable monomers (31) of this type are disclosed in DE-A 196 25 773 A1, column 3, line 65, to column 4, line 20). These may comprise minor amounts of higher-functional alkyl or cycloalkyl (meth)acrylates, such as ethylene glycol, propylene glycol, diethylene glycol, dipropylene glycol, butylene glycol, pentane-1,5-diol, hexane-1,6-diol, octahydro-4,7-methano-1H-indenedimethanol or cyclohexane-1,2-, -1,3- or -1,4-diol di(meth)acrylate; trimethylolpropane di- or tri(meth)acrylate; or pentaerythrityl di-, tri- or tetra(meth)-acrylate. In the context of the present invention, minor amounts of higher-functional monomers (a31) are to be understood here as meaning those amounts which do not lead to crosslinking or gelling of the copolymers, unless they are to be present in the form of crosslinked particles.
(a32) Vinyl esters of monocarboxylic acids branched in the alpha-position and having 5 to 18 carbon atoms in the molecule. The branched monocarboxylic acids may be obtained by reacting formic acid or carbon monoxide and water with olefins in the presence of a liquid, strongly acidic catalyst; the olefins may be crack products of paraffinic hydrocarbons, such as mineral oil fractions, and may comprise both branched and straight-chain acyclic and/or cycloaliphatic olefins. In the reaction of such olefins with formic acid or with carbon monoxide and water, a mixture of carboxylic acids forms, in which the carboxyl groups are predominantly located on a quaternary carbon atom. Other olefinic starting materials are, for example, propylene trimers, propylene tetramers and diisobutylene. The vinyl esters can, however, also be prepared in a manner known per se from the acids, for example by allowing the acid to react with acetylene. Owing to the good availability, vinyl esters of saturated aliphatic monocarboxylic acids having 9 to 11 carbon atoms, which are branched at the alpha-carbon atom, are particularly preferably used. Vinyl esters of this type are sold under the brand VeoVa® (cf. also Römpp Lexikon Lacke and Druckfarben, Georg Thieme Verlag, Stuttgart, New York, 1998, page 598).
(a33) Diarylethylenes, in particular those of the general formula I:

R¹R²C═CR³R⁴ (I)
where the radicals R¹, R², R³ and R⁴, in each case independently of one another, are hydrogen atoms or substituted or unsubstituted alkyl, cycloalkyl, alkylcycloalkyl, cycloalkylalkyl, aryl, alkylaryl, cycloalkylaryl, arylalkyl or arylcycloalkyl radicals, with the proviso that at least two of the variables R¹, R², R³ and R⁴ are substituted or unsubstituted aryl, arylalkyl or arylcycloalkyl radicals, in particular substituted or unsubstituted aryl radicals. Examples of suitable alkyl radicals are methyl, ethyl, propyl, isopropyl, n-butyl, isobutyl, tert-butyl, amyl, hexyl or 2-ethylhexyl. Examples of suitable cycloalkyl radicals are cyclobutyl, cyclopentyl or cyclohexyl. Examples of suitable alkylcycloalkyl radicals are methylenecyclohexane, ethylenecyclohexane or propane-1,3-diylcyclohexane. Examples of suitable cyclo-alkylalkyl radicals are 2-, 3- or 4-methyl, -ethyl-, -propyl- or -butylcyclohex-1-yl. Examples of suitable aryl radicals are phenyl, naphthyl or biphenylyl, preferably phenyl and naphthyl and in particular phenyl. Examples of suitable alkylaryl radicals are benzyl or ethylene- or propane-1,3-diylbenzene. Examples of suitable cycloalkylaryl radicals are 2-, 3- or 4-phenylcyclohex-1-yl. Examples of suitable arylalkyl radicals are 2-, 3- or 4-methyl-, -ethyl-, -propyl- or -butylphen-1-yl. Examples of suitable arylcycloalkyl radicals are 2-, 3- or 4-cyclohexylphen-1-yl. The aryl radicals R¹, R², R³ and/or R⁴ are preferably phenyl or naphthyl radicals, in particular phenyl radicals. The substituents present, if appropriate, in the radicals R¹, R², R³ and/or R⁴ are electron-retracting or electron-repelling atoms or organic radicals, in particular halogen atoms, nitrile radicals, nitro radicals, partially or completely halogenated alkyl, cycloalkyl, alkylcycloalkyl, cycloalkylalkyl, aryl, alkylaryl, cycloalkyl-aryl, arylalkyl and arylcycloalkyl radicals; aryloxy, alkoxy and cycloalkoxy radicals; and/or arylthio, alkylthio and cycloalkylthio radicals. Diphenylethylene, dinaphthalene-ethylene, cis- or trans-stilbene or vinylidene-bis(4-nitrobenzene) are particularly advantageous, especially diphenylethylene (DPE), which is why they are preferably used. In the present invention, the monomers (b33) are used for regulating the copolymerization in an advantageous manner in such a way that a free radical copolymerization in the batch procedure is also possible.
(a34) Vinylaromatic hydrocarbons, such as styrene, vinyltoluene, diphenylethylene or alpha-alkyl-styrenes, in particular alpha-methylstyrene.
(a35) Nitriles, such as acrylonitrile and/or methacrylonitrile.
(a36) Vinyl compounds, in particular vinyl and/or vinylidene dihalides, such as vinyl chloride, vinyl fluoride, vinylidene dichloride or vinylidene difluoride; N-vinylamides, such as vinyl-N-methylformamide, N-vinylcaprolactam or N-vinylpyrrolidone; 1-vinylimidazole; vinyl ethers, such as ethyl vinyl ether, n-propyl vinyl ether, isopropyl vinyl ether, n-butyl vinyl ether, isobutyl vinyl ether and/or vinyl cyclohexyl ether; and/or vinyl esters, such as vinyl acetate, vinyl propionate, vinyl butyrate, vinyl pivalate and/or the vinyl ester of 2-methyl-2-ethylheptanoic acid.
(a37) Allyl compounds, in particular allyl ethers and esters, such as allyl methyl, ethyl, propyl or butyl ether, or allyl acetate, propionate or butyrate.
(a38) Polysiloxane macromonomers which have a number average molecular weight Mn of from 1000 to 40 000 and on average from 0.5 to 2.5 ethylenically unsaturated double bonds per molecule; in particular polysiloxane macromonomers which have a number average molecular weight Mn of from 2000 to 20 000, particularly preferably from 2500 to 10 000 and in particular from 3000 to 7000 and on average from 0.5 to 2.5, preferably from 0.5 to 1.5, ethylenically unsaturated double bonds per molecule, as described in DE 38 07 571 A 1 on pages 5 to 7, in DE 37 06 095 A 1 in columns 3 to 7, in EP 0 358 153 B1 on pages 3 to 6, in U.S. Pat. No. 4,754,014 A 1 in columns 5 to 9, in DE 44 21 823 A 1 or in the international patent application WO 92/22615 on page 12, line 18, to page 18, line 10; and
(a39) olefins, such as ethylene, propylene, but-1-ene, pent-1-ene, hex-1-ene, cyclohexene, cyclo-pentene, norbornene, butadiene, isoprene, cyclopentadiene and/or dicyclopentadiene.

It is of course possible to choose for the preparation of the (meth)acrylate copolymers and the above-mentioned monomers (a) in such a way that the (meth)acrylate copolymers (A) comprise an amount of (meth)acrylate monomers (a) incorporated in the form of polymerized units so that their property profile is determined primarily by these monomers. In general, this is the case at a content of >50 mol %, preferably >60 mol %, based in each case on the (meth)acrylate copolymer (A).

For the preparation of the butadiene copolymers (A), the weight ratio of butadiene (a) to comonomers (a) can be very widely varied and may be, for example in the case of vinylaromatic comonomers (a34), from 10:90 to 90:10, preferably from 20:80 to 80:20.

Higher-functional monomers (a) of the type described above are generally used in minor amounts. In the context of the present invention, minor amounts of higher-functional monomers are to be understood as meaning those amounts which do not lead to crosslinking or gelling of the (meth)acrylate copolymers or of the butadiene copolymers (A), unless it is intended to prepare crosslinked polymer particles (A).

The (meth)acrylate copolymers and butadiene copolymers (A) can be prepared by the customary and known mass, solution or emulsion copolymerization processes. They are preferably prepared with the aid of free radical emulsion polymerization because they are obtained thereby in the form of dispersed polymer particles (A) which can be used directly for the preparation of the polymer dispersion according to the invention.

Dispersions of such polymer particles (A) are customary and known products and are sold, for example, by BASF AG under the brands Styronal© and Acronal® for paper conversion.

The polymer dispersion according to the invention comprises, based in each case on its solids, from 1 to 20% by weight, preferably from 1.5 to 15% by weight and in particular from 2 to 10% by weight of at least one, in particular one, type of water-dispersible polymer particles (B) which comprise at least one fluorescent dye.

The fluorescent dye or the fluorescent dyes preferably is or are distributed in molecular disperse form in the matrix of the polymer particles (B). Preferably, the fluorescent dyes have a very much higher solubility in the polymer matrix (B) than in the continuous aqueous phase of the polymer dispersion according to the invention. The content of fluorescent dyes in the polymer particles may vary widely and can therefore be optimally adapted to the requirements of the individual case. Preferably, the content, based in each case on the total amount of the polymer particles (B), is from 0.01 to 10% by weight and in particular from 0.01 to 1% by weight.

Fluorescent dyes are known to be organic colorants whose optical effect is based on their ability to absorb UV radiation or visible light and to emit it as light of greater wavelength with a time lag (phosphorescence) or without a time lag (fluorescence) (cf. DIN 55944 2003-11).

Examples of suitable fluorescent dyes are disclosed in European patent application EP 1 801 127 A1, page 5, paragraph [0023].

The particle size of the polymer particles (B) may vary widely and can therefore be optimally adapted to the requirements of the individual case. In a given polymer dispersion according to the invention, the particle size of the polymer particles (B) is preferably greater than the particle size of the polymer particles (A). The polymer particles (B) preferably have a volume-average particle size of from 1 to 100 μm, preferably from 1.1 to 25 μm and in particular from 1.1 to 6 μm, determined by light scattering.

The polymer particles (B) or their aqueous dispersions can be prepared by a very wide range of customary and known methods. They are preferably obtained by the free radical suspension polymerization of ethylenically unsaturated monomers in an oil-in-water emulsion whose disperse phase (oil phase) comprises at least one fluorescent dye in solution and has a mean particle size, determined by light scattering, of at least 1 μm.

A fluorescent dye which has a very much higher solubility in the oil phase than in the continuous aqueous phase of the oil-in-water emulsion is preferably used here. The solubility of the fluorescent dyes in the oil phase is preferably >0.001% by weight, in particular >0.01% by weight, based in each case on the total amount of the oil phase.

Suitable ethylenically unsaturated monomers (b) for the preparation of the polymer particles (B) by the free radical suspension polymerization are the ethylenically unsaturated monomers (a) described above.

Further examples of suitable ethylenically unsaturated monomers (b) are disclosed in European patent application EP 1 801 127 A1, page 4, paragraph [0014], to page 5, paragraph [0025]. The free radical suspension polymerization is preferably carried out with at least one of the hydrophobic ethylenically unsaturated monomers (a) which are described there and which are referred to here as monomers (b1) for reasons of systematics. In particular, the free radical suspension polymerization is carried out using a mixture which is described there and which comprises or consists of
(a) at least one hydrophobic ethylenically unsaturated monomer, referred to here as monomer (b1) for reasons of systematics, and
(b) at least one hydrophilic ethylenically unsaturated monomer, referred to here as monomer (b2) for reasons of systematics, and/or
(c) at least one crosslinking monomer having at least two, in particular two, ethylenically unsaturated double bonds, referred to here as monomer (b3) for reasons of systematics.

Particularly advantageous ratios are likewise disclosed in the European patent application EP 1 801 127 A1, page 5, paragraphs [0021] and [0022].

Preferably, the free radical suspension polymerization of the monomers (b) is carried out in the presence of at least one hydrophobic substance which stabilizes the oil-in-water emulsion and significantly increases the solubility of the fluorescent dyes in the oil phase and the polymer particles (B) formed therefrom. The hydrophobic substance is then present as a stabilizing additive (D) in the polymer dispersion according to the invention. Preferably, the hydrophobic substance has a solubility in water of <0.01 g/l at 25° C. and 1013 hectopascal. Its amount may be widely varied and can therefore be optimally adapted to the requirements of the individual case. It is preferably used in an amount of from 1 to 20% by weight, based on the monomers (b). Examples of suitable hydrophobic substances are likewise disclosed in European patent application EP 1 801 127 A1, page 5, paragraph [0025], to page 6, paragraph [0033].

The free radical suspension polymerization of the monomers (b) is preferably carried out in the presence of at least one customary and known surface-active compound which serves for stabilizing the oil-in-water emulsion and which is then present as stabilizing additive (D) in the polymer dispersion according to the invention.

The content of surface-active compound (D) in the oil-in-water emulsion may vary widely and can therefore be optimally adapted to the requirements of the individual case. Preferably, it is used in an amount of up to 15% by weight, preferably from 0.05 to 15% by weight, particularly preferably from 0.05 to 5% by weight and in particular from 0.1 to 2% by weight, based in each case on the total oil-in-water emulsion. It is preferably added to the aqueous phase prior to emulsification.

Examples of suitable surface-active compounds (D) are anionic and/or nonionic surfactants, amphiphilic polymers, polymers of monoethylenically unsaturated acids, graft copolymers of N-vinylformamide on polyalkylene glycols and zwitterionic polyalkylene polyamines, as disclosed, for example, in the European patent application EP 1 801 127 A1, page 6, paragraph [0034], to page 8, paragraph [0043].

The free radical suspension polymerization of the monomers (b) is also preferably carried out in the presence of at least one, in particular one, protective colloid which likewise serves for stabilizing the oil-in-water emulsion and which is then present as stabilizing additive (D) in the polymer dispersion according to the invention. Examples of suitable protective colloids are disclosed in Römpp Online 2007, “Schutzkolloid” or in the European patent application EP 1 801 127 A1, page 8, paragraphs [0044] to [0046].

In the free radical suspension polymerization of the monomers (b), it is also possible to use at least one customary and known polymerization regulator, preferably in an amount of from 0.01 to 5% by weight and in particular from 0.1 to 1% by weight, based in each case on the monomers (b). Examples of suitable polymerization regulators are disclosed in the European patent application EP 1 801 127 A1, page 8, paragraph [0047].

The free radical suspension polymerization of the monomers (b) is usually initiated by at least one, in particular one, customary and known free radical polymerization initiator. Suitable polymerization initiators are all compounds which can initiate a polymerization, such as, for example, peroxides, hydroperoxides, azo compounds and redox catalysts, as disclosed in the International patent application WO 99/40123, page 32, line 45, to page 34, line 9.

In addition or alternatively to the polymerization initiators, the free radical suspension polymerization of the monomers (b) can also be initiated by the action of actinic radiation, the procedure being effected, if appropriate, in the presence of at least one customary and known, preferably oil-soluble, sensitizer. In addition, the free radical suspension polymerization can also be initiated electrochemically, by ultrasound or by microwave radiation.

The preparation of the oil-in-water emulsions has no peculiarities in terms of the method, but rather is effected in a customary and known manner, for example by dissolving at least one fluorescent dye in a mixture of at least one ethylenically unsaturated monomer (b) and at least one hydrophobic substance and emulsifying the resulting solution in an aqueous phase which comprises at least one surface-active compound. The customary and known mixing units, such as high-speed stirrers, Ultraturrax, inline dissolvers, homogenizing nozzles, static mixers or microfluidizers, can be used here. Stabilized oil-in-water emulsions having a mean particle size of the oil droplets of at least 1 to 100 μm, preferably from 1.1 to 25 μm and in particular from 1.1 to 6 μm result.

The amount of the oil phase is preferably such that a dispersion of polymer particles (B) having a solids content of from 10 to 60% by weight, preferably from 20 to 45% by weight and in particular from 30 to 45% by weight results.

The temperature at which the free radical suspension polymerization is carried out may vary widely and can thus be adapted to the requirements of the individual case in an outstanding manner. Said suspension polymerization is preferably carried out at from 0 to 120° C., it being carried out at temperatures of >100° C. under superatmospheric pressure in a pressure-tight reaction vessel.

The polymer dispersion according to the invention comprises, as a further substantial component, at least one, in particular one, thickener (C) in an amount of from 0.001 to 3% by weight, in particular from 0.02 to 0.3% by weight, based in each case on the solids of the polymer dispersion.

In principle, all customary and known thickeners (C) are suitable for use in the polymer dispersion according to the invention, provided that they do not undergo with the other constituents undesired interactions which lead, for example, to the formation of flocs.

The thickener (C) is preferably selected from the group consisting of pyrogenic silicas, phyllosilicates, low molecular weight organic natural products and synthetic products, polymeric and oligomeric organic natural products and modified natural products, fully synthetic, polymeric and oligomeric organic thickeners without a surfactant character and associative thickeners having a surfactant character (cf. Römpp Online 2007, “Verdickungsmittel”).

Examples of suitable phyllosilicates (C) are smectites, in particular montmorillonites and hectorites and reaction products of smectites with quaternary ammonium compounds, i.e. hydrophobic organophyllosilicates.

Examples of suitable low molecular weight organic natural products and synthetic products (C) are metal soaps, hydrogenated castor oil, modified fat derivatives and polyamides.

Examples of suitable polymeric and oligomeric organic natural products (C) are starch, gelatin, casein, gum arabic, xanthan and gellan.

Examples of suitable polymeric and oligomeric modified organic natural products (C) are hydroxyethylcellulose (HEC), carboxymethylcellulose (CMC), hydroxypropyl-methylcellulose (HPMC), hydroxypropylcellulose (HPC) and ethylhydroxyethylcellulose (EHEC).

Examples of suitable fully synthetic thickeners (C) without a surfactant character which are not capable of forming micelles are polyvinyl alcohols, polyacrylic acids and polymethylacrylic acids and salts thereof, polyacrylamides, polyvinylpyrrolidone, polyethylene glycols, styrene/maleic anhydride copolymers and salts thereof and copolymers of acrylic acid and/or methacrylic acid and salts thereof.

Examples of suitable associative thickeners (C) having a surfactant character are hydrophobically modified polyacrylates (“hydrophobically modified alkali swellable emulsions”, HASE), hydrophobically modified cellulose ethers (“hydrophobically modified hydroxy-ethyl cellulose”, HMHEC), hydrophobically modified polyacrylamides (HMPAM), hydrophobically modified polyethers (HMP) and associative polyurethane thickeners.

The thickener (C) is preferably selected from the group consisting of fully synthetic polymeric and oligomeric thickeners without a surfactant character and polymeric and oligomeric organic natural products and modified natural products.

In particular, the fully synthetic polymeric and oligomeric thickeners (C) without a surfactant character are copolymers and terpolymers of acrylic acid and/or methacrylic acid with at least one further ethylenically unsaturated monomer, and the polymeric and oligomeric organic natural products and modified natural products are polysaccharides.

The particularly preferably used fully synthetic copolymers (C) of acrylic acid and/or methacrylic acid with at least one further ethylenically unsaturated monomer are customary and known compounds and are preferably sold in the form of their aqueous dispersions under the brand Sterocoll® by BASF AG or under the brand Viskalex® HV 30 by Ciba Specialty Chemicals.

The particularly preferably used polysaccharides (C) are xanthan or gellan.

Apart from the substantial constituents (A), (B) and (C) described above, the polymer dispersion according to the invention may also comprise at least one stabilizing additive (D) in effective amounts. The stabilizing additive (D) is preferably selected from the group consisting of the above-described hydrophobic substances, surface-active compounds and protective colloids and of compositions for adjusting the pH, light stabilizers, UV absorbers, low- and high-boiling organic solvents, polymerization inhibitors and antifoams.

Hydrophobic substances, surface-active compounds and protective colloids are preferably used as stabilizing additives (D). In particular they are introduced into the polymer dispersion according to the invention via the aqueous dispersions of the polymer particles (B).

The polymer dispersion according to the invention can be prepared by any suitable process. It is preferably prepared by the preparation process I according to the invention by homogeneously mixing, based on the solids of the polymer dispersion,
(A) from 1 to 99% by weight of at least one type of water-soluble and/or water-dispersible, in particular dispersible, polymer,
(B) from 1 to 20% by weight of at least one type of water-dispersible polymer particles which comprise at least one fluorescent dye and
(C) from 0.001 to 3% by weight of at least one thickener and
(D) if appropriate, at least one additive stabilizing the polymer dispersion
   in water.

The polymers (A) are preferably used in the form of an aqueous solution (A) or aqueous dispersion (A), in particular of an aqueous dispersion (A), and the polymer particles (B) in the form of an aqueous dispersion (B).

The solution (A) or the dispersion (A), in particular the dispersion (A), is particularly preferably initially taken, after which the dispersion (B) is added to the initially taken solution or dispersion.

The thickener (C) can be added to the solution or dispersion (A) and/or to the dispersion (B) prior to the mixing thereof; it is preferably added to the mixture of the solution (A) or the dispersion (A) with the dispersion (B).

At least one further stabilizing additive (D), preferably a composition for adjusting the pH, can then also be added to the polymer dispersion according to the invention. The pH of the polymer dispersion according to the invention is more preferably adjusted to 7 to 9, in particular 7 to 8.

For the preparation process I according to the invention, the mixing units described above can be used.

The solids content of the polymer dispersion according to the invention may vary very widely and can thus be adapted in an outstanding manner to the requirements of the respective intended use. Preferably, the solids content is from 10 to 60% by weight, preferably from 15 to 50% by weight, and in particular from 20 to 40% by weight.

The resulting polymer dispersion according to the invention shows no settling on storage at rest for at least 1.5 months, preferably for at least 2 months and in particular for at least 6 months. This can be demonstrated in a particularly simple manner by storing the polymer dispersion according to the invention in a standing cylinder. If polymer particles were to settle, this could be detected very accurately visually or by the spectroscopic determination of the concentration gradient of the fluorescent dye as a function of the height of the liquid column.

In the use according to the invention, the polymer dispersions according to the invention can be used as such as marked, liquid functional products, in particular as novel marking materials, coating materials, adhesives, sealing compounds, printing inks, paper coating slips, paper sizes or starting materials for the production of shaped articles and films.

The novel liquid marking materials can be used, for example, for the marking of liquids, such as fuels. The novel liquid coating materials, adhesives, sealing compounds, printing inks, paper coating slips, paper sizes and starting materials for the production of shaped articles and films can advantageously be used for the production of novel marked coatings, adhesive layers, seals, printed substrates, converted paper products, shaped articles and films.

The polymer dispersions according to the invention are preferably used for the production of marked, liquid functional products, marked, solid pulverulent functional products and of marked, solid compact functional products which comprise at least one effect-imparting additive (E). These functional products may be not only solid or liquid marking materials, coating materials, adhesives, sealing compounds, printing inks, paper coating slips, paper sizes and starting materials for the production of shaped articles and films but also catalysts, formulations of crop protection agents and cosmetic and pharmaceutical formulations.

The choice of the effect-imparting additives (E) depends on the respective intended use of the novel functional products. The effect-imparting additive (E) is preferably selected from the group consisting of crosslinking agents which are crosslinkable thermally and/or with actinic radiation; reactive diluents which are heat-curable and/or curable with actinic radiation; dyes which differ from the fluorescent dyes and are soluble in water and/or in the polymer particles (A) and/or (B); slip additives; catalysts for thermal crosslinking; thermolabile free radical initiators; photoinitiators; adhesion promoters; leveling agents; film-forming assistants; flameproofing agents; corrosion inhibitors; flow improvers; waxes; siccatives; biocides; dulling agents; deaerating agents; organic or inorganic, opaque and transparent pigments; metal powders; organic and inorganic, transparent and opaque fillers; organic and inorganic nanoparticles; active substances of crop protection agents; pharmaceuticals and fragrances.

The effect-imparting additive (E) is preferably selected from the group consisting of organic or inorganic, opaque and transparent pigments, in particular colored pigments, effect pigments, electrically conductive pigments and magnetically shielding pigments; metal powders; organic and inorganic, transparent and opaque fillers and organic and inorganic nanoparticles.

Examples of suitable effect pigments (E) are lamellar metal pigments, such as commercially available aluminum bronzes, aluminum bronzes chromatized according to DE 36 36 183 A1, and commercially available stainless steel bronzes and nonmetallic effect pigments, such as, for example, pearl luster or interference pigments, lamellar effect pigments based on iron oxide, which has a hue from pink to brown-red, or liquid crystalline effect pigments. In addition, reference is made to Römpp Lexikon Lacke and Druckfarben, Georg Thieme Verlag, 1998, page 176, “Effektpigmente”, and pages 380 and 381, “Metalloxid-Glimmer-Pigmente” to “Metallpigmente”, and the patent applications and patents DE 36 36 156 A1, DE 37 18 446 A1, DE 37 19 804 A1, DE 39 30 601 A1, EP 0 068 311 A1, EP 0 264 843 A1, EP 0 265 820 A1, EP 0 283 852 A1, EP 0 293 746 A1, EP 0 417 567 A1, U.S. Pat. No. 4,828,826 A or U.S. Pat. No. 5,244,649 A.

Examples of suitable inorganic color-imparting pigments (E) are white pigments, such as titanium dioxide, zinc white, zinc sulfide or lithopone; black pigments, such as carbon black, iron manganese black or spinel black; color pigments, such as chromium oxide, chromium oxide hydrate green, cobalt green or ultramarine green, cobalt blue, ultramarine blue or manganese blue, ultramarine violet or cobalt and manganese violet, iron oxide red, cadmium sulfoselenide, molybdate red or ultramarine red; iron oxide brown, mixed brown, spinel and corundum phases or chrome orange; or iron oxide yellow, nickel titanium yellow, chromium titanium yellow, cadmium sulfide, cadmium zinc sulfide, chrome yellow or bismuth vanadate.

Examples of suitable organic color-imparting pigments (E) are monoazo pigments, bisazo pigments, anthraquinone pigments, benzimidazole pigments, quinacridone pigments, quinophthalone pigments, diketo-pyrrolopyrrole pigments, dioxazine pigments, indanthrone pigments, isoindoline pigments, isoindolinone pigments, azomethine pigments, thioindigo pigments, metal complex pigments, perinone pigments, perylene pigments, phthalocyanine pigments or aniline black.

In addition, reference is made to Römpp Lexikon Lacke und Druckfarben, Georg Thieme Verlag, 1998, pages 180 and 181, “Eisenblau-Pigmente” to “Eisenoxidschwarz”, pages 451 to 453, “Pigmente” to “Pigmentvolumenkonzentration”, page 563, “Thioindigo-Pigmente”, page 567, “Titandioxid-Pigmente”, pages 400 and 467, “Natürlich vorkommende Pigmente”, page 459, “Polycyclische Pigmente”, page 52, “Azomethin-Pigmente”, “Azopigmente”, and page 379, “Metallkomplex-Pigmente”.

Examples of suitable electrically conductive pigments (E) are titanium dioxide/tin oxide pigments.

Examples of magnetically shielding pigments (E) are pigments based on iron oxides and chromium dioxide.

Examples of suitable metal powders (E) are powders of metals and metal alloys aluminum, zinc, copper, bronze or brass.

Examples of suitable organic and inorganic fillers (E) are chalk, calcium sulfates, barium sulfate, silicates, such as talc, mica or kaolin, silicas, oxides, such as aluminum hydroxide or magnesium hydroxide, or organic fillers, such as plastic powders, in particular comprising polyamide or polyacrylonitrile. In addition, reference is made to Römpp Lexikon Lacke und Druckfarben, Georg Thieme Verlag, 1998, page 250 et seq., “Füllstoffe”.

Examples of suitable transparent fillers (E) are those based on silica, alumina or zirconium oxide.

Suitable nanoparticles (E) are preferably selected from the group consisting of metals, compounds of metals and organic compounds.

The metals are preferably selected from the third to fifth main group and the third to sixth and the first and second subgroup of the Periodic Table of the Elements and the lanthanides, and preferably from the group consisting of boron, aluminum, gallium, silicon, germanium, tin, arsenic, antimony, silver, zinc, titanium, zirconium, hafnium, vanadium, niobium, tantalum, molybdenum, tungsten and cerium. In particular, aluminum and silicon are used.

The compounds of the metals are preferably oxides, hydrated oxides, sulfates, hydroxides or phosphates, in particular oxides, hydrated oxides and hydroxides.

Examples of suitable organic compounds are lignins and starches.

Nanoparticles (E) preferably have a primary particle size of <50, preferably from 5 to 50, in particular from 10 to 30, nm.

The novel, marked, liquid, solid pulverulent and solid compact functional products which comprise at least one effect-imparting additive (E) can be prepared by customary and known suitable processes. According to the invention, it is, however, advantageous to prepare them by the preparation process II according to the invention.

The preparation process II according to the invention starts from a polymer dispersion according to the invention, in particular a polymer dispersion according to the invention which is prepared by the preparation process I according to the invention. The polymer dispersion according to the invention is homogeneously mixed with at least one of the effect-imparting additives (E) described above. The effect-imparting additive (E) can be added in its total amount or in portions to the polymer dispersion according to the invention. Alternatively, the effect-imparting additives (E) can be added in their total amount or in portions or in succession in their respective total amount or in portions to the polymer dispersion according to the invention. Conversely, the polymer dispersion according to the invention can be added in its total amount or in portions to the effect-imparting additive (E) or the effect-imparting additives (E). The mixing units described above can be used thereby.

In the preparation process II according to the invention, marked, liquid functional products which comprise at least one effect-imparting additive (E) and can be used as such for the production of end products, such as, for example, marked fuels, coatings, adhesive layers, seals, printed substrates, converted paper products, films, shaped articles, catalysts, crop protection agents, cosmetics and drugs, result.

The marked, liquid functional products which result in the preparation process II according to the invention and comprise at least one effect-imparting additive (E) can, however, also be intermediates which serve for the production of marked, solid pulverulent and solid compact functional products. This can be effected by drying the liquid functional products, for example by freeze-drying.

The resulting marked, solid pulverulent functional products can be converted in at least one further process step into marked, solid compact functional products, such as films and moldings. This can be effected, for example, by melting the marked, solid pulverulent functional products and blowing them to give films or casting them in molds and then curing them physically or thermally and/or with actinic radiation.

The functional products described above and the end products produced from them have outstanding performance characteristics.

### EXAMPLES AND COMPARATIVE EXPERIMENT

### Preparation Examples 1 to 8

### The preparation of the aqueous dispersions 1 to 8, comprising the polymer particles (B1) to (B8)

For the examples and the comparative experiment, the aqueous dispersions 1 to 8, comprising the polymer particles (B1) to (B8), were first prepared.

### Preparation Example 1

### Aqueous Dispersion 1 Comprising the Polymer Particles (B1)

The following reaction mixture was initially taken in a 2 l vessel having a dispenser stirrer (diameter 5 cm):

450 g of water,
250 g of polyvinyl alcohol [Mowiol ® 40/88 (10%
strength in water)],
2.1 g of sodium nitrite (2.5% strength in water),
30 g of 1,4-butanediol diacrylate,
270 g of methyl methacrylate,
0.09 g of red fluorescent dye (Lumogen ® F-red) and
15 g of white oil (CAS No. 8042-47-5).

The reaction mixture was dispersed for 30 minutes at room temperature at a speed of 5000 rpm and then transferred to a 2 l vessel equipped with an anchor stirrer. 2.1 g of tert-butyl perpivalate (75% strength, dissolved in isododecane) were added and the reaction mixture was heated to 60° C. in the course of one hour. The reaction mixture was then heated to 70° C. in the course of two hours. Thereafter, the temperature was increased to 85° C. for 30 minutes and the reaction mixture was kept at this temperature for one hour. 7 g of a 10% strength aqueous solution of tert-butyl hydroperoxide were then added. A solution of 0.4 g of ascorbic acid in 20 g of water was then metered in the course of one hour. The reaction mixture was then cooled to room temperature.

The aqueous dispersion 1 of the crosslinked polymer particles (B1) marked with the fluorescent dye and having a volume-average particle size of 1.3 μm and a broad particle size distribution (measured with the aid of light scattering analysis according to the Mie theory using a Malvern Mastersizer®, a refractive index of 1.49 and an absorption index of 0 being taken as a basis for the particles) resulted. The solids content was 27.6% by weight.

### Preparation Example 2

### Aqueous Dispersion 2 Comprising the Polymer Particles (B2)

The following reaction mixture was initially taken in a 2 l vessel having a dispenser stirrer (diameter 5 cm):

450 g of water,
250 g of polyvinyl alcohol [Mowiol ® 40/88 (10%
strength in water)],
2.1 g of sodium nitrite (2.5% strength in water),
30 g of 1,4-butanediol diacrylate,
270 g of methyl methacrylate,
0.09 g of red fluorescent dye (Lumogen ® F-red) and
15 g of hexadecane.

Emulsification of the reaction mixture and its suspension polymerization were effected as described in preparation example 1.

The aqueous dispersion 2 of the crosslinked polymer particles (B2) marked with the fluorescent dye and having a volume-average particle size of 1.9 μm and a broad particle size distribution resulted. The solids content was 33.7% by weight.

### Preparation Example 3

### Aqueous Dispersion 3 Comprising the Polymer Particles (B3)

The following reaction mixture was initially taken in a 2 l vessel having a dispenser stirrer (diameter 5 cm):

450 g of water,
250 g of polyvinyl alcohol [Mowiol ® 40/88 (10%
strength in water)],
2.1 g of sodium nitrite (2.5% strength in water),
30 g of allyl methacrylate,
270 g of methyl methacrylate,
0.06 g of yellow fluorescent dye (Lumogen ® F-yellow
083) and
15 g of hexadecane.

Emulsification of the reaction mixture and its suspension polymerization were effected as described in preparation example 1.

The aqueous dispersion 3 of the crosslinked polymer particles (B3) marked with the fluorescent dye and having a volume-average particle size of 2 μm and a narrow particle size distribution having a uniformity of 0.5 resulted. The solids content was 30.85% by weight.

### Preparation Example 4

### Aqueous Dispersion 4 Comprising the Polymer Particles (B4)

The following reaction mixture was initially taken in a 2 l vessel which was equipped with a dispenser stirrer (diameter 5 cm):

441.45 g of water,
45 g of polyvinyl alcohol (Mowiol ® 15/79 (10% in
water)),
180 g of methylhydroxypropylcellulose Culminal ® MHPC
100 (5% in water),
52.5 mg of NaNO₂,
30 g of butanediol diacrylate,
270 g of methyl methacrylate,
0.06 g of yellow fluorescent dye (Lumogen ® F-yellow
083) and
15 g of hexadecane.

The emulsification of the reaction mixture and its suspension polymerization were effected as described in preparation example 1. A dispersion 4 comprising crosslinked polymer particles (B4) marked with fluorescent dye and having a mean particle diameter of 2.6 μm and a narrow particle size distribution with a uniformity of 0.5 was obtained. The solids content of the dispersion 4 was 29.6% by weight.

### Preparation Example 5

### Aqueous Dispersion 5 Comprising the Polymer Particles (B5)

The following reaction mixture was initially taken in a 2 l vessel having a dispenser stirrer (diameter 5 cm):

502.7 g of water,
30 g of polyvinyl alcohol [Mowiol ® 15/79 (10% in
water)],
120 g of methylhydroxypropylcellulose Culminal ® MHPC
100 (5% in water),
52.5 mg of NaNO₂,
120 g of butanediol diacrylate,
150 g of methyl methacrylate,
30 g of methacrylic acid,
0.06 g of yellow fluorescent dye (Lumogen ® F-yellow
083),
0.015 g of hydroquinone monomethyl ether and
15 g of hexadecane.

The emulsification of the reaction mixture and its suspension polymerization were effected as described in preparation example 1.

A dispersion 5 comprising crosslinked polymer particles (B5) marked with fluorescent dye and having a mean particle diameter of 4.9 μm was obtained. The solids content of the dispersion 5 was 30.3% by weight.

### Preparation Example 6

### Aqueous Dispersion 6 Comprising the Polymer Particles (B6)

The following reaction mixture was initially taken in a 2 l vessel having a dispenser stirrer (diameter 5 cm):

450 g of water,
250 g of polyvinyl alcohol [Mowiol ® 40/88 (10% in
water)],
2.1 g of NaNO₂ (2.5% in water),
30 g of allyl methacrylate,
240 g of methyl methacrylate,
30 g of n-butyl acrylate,
0.09 g of red fluorescent dye (Lumogen ® F-red 305)
15 g of hexadecane.

The emulsification of the reaction mixture and its suspension polymerization were effected as described in preparation example 1.

A dispersion 6 comprising crosslinked polymer particles (B6) marked with fluorescent dye and having a mean particle diameter of 2.1 μm and a uniformity of 0.4 was obtained. 6 g of coagulum were found and the solids content of the dispersion 6 was 29.9% by weight.

### Preparation Example 7

### Aqueous Dispersion 7 Comprising the Polymer Particles (B7)

The following reaction mixture was initially taken in a 2 l vessel having a dispenser stirrer (diameter 5 cm):

370.1 g of water,
208.3 g of polyvinyl alcohol [Mowiol ® 40/88 (10% in
water)],
1.75 g of NaNO₂ (2.5% in water),
25 g of butanediol diacrylate,
225 g of methyl methacrylate,
0.75 g of ethylhexyl thioglycolate,
0.08 g of red fluorescent dye (Lumogen ® F-red 305) and
12.5 g of hexadecane.

The emulsification of the reaction mixture and its suspension polymerization were effected as described in preparation example 1.

A dispersion 7 comprising crosslinked polymer particles (B7) marked with fluorescent dye and having a mean particle diameter of 1.6 μm and a uniformity of 0.4 was obtained, and the solids content of the dispersion 7 was 28.3% by weight.

### Preparation Example 8

### Aqueous Dispersion 8 Comprising the Polymer Particles (B8)

The following reaction mixture was initially taken in a 2 l vessel having a dispenser stirrer (diameter 5 cm):

450 g of water,
250 g of polyvinyl alcohol [Mowiol ® 40/88 (10% in
water)],
2.1 g of NaNO₂ (2.5% in water),
300 g of methyl methacrylate,
0.09 g of red fluorescent dye (Lumogen ® F-red 305) and
15 g of hexadecane.

The emulsification of the reaction mixture and its suspension polymerization were effected as described in preparation example 1, the total amount of tert-butyl perpivalate being divided into three equal parts. The first part was added at 60° C., the second part at 65° C. and the third part at 70° C.

A dispersion 8 comprising uncrosslinked polymer particles (B8) marked with fluorescent dye and having a mean particle diameter of 1.5 μm and a uniformity of 0.4 was obtained, and the solids content of the dispersion 8 was 31.3% by weight.

### Comparative Experiment C1

### The Preparation of an Aqueous Dispersion C1 Comprising the Polymer Particles (B1)

1.6 kg of an aqueous dispersion of an acrylate copolymer (A) (Acronal® S 728 from BASF AG) were initially taken in a mixing pot. 0.103 kg of the aqueous dispersion 1 of preparation example 1 was added to the initially taken aqueous dispersion with thorough stirring.

The Brookfield viscosity (spindle No. 2) of the resulting aqueous dispersion C1 was 312 mPa·s.

The aqueous dispersion C1 was introduced into a standing cylinder with a height of fill of 10 cm. The settling behavior was assessed visually: after only 3 weeks, settling of the colored polymer particles (B1) was observable.

### Examples 1 to 8

The preparation of the pigment-free, aqueous polymer dispersions 1 to 8 marked with fluorescent dyes and comprising the polymer particles (B1), (B2) or (B3) and thickener (C)

### General Experimental Method:

In each case an aqueous dispersion of an acrylate copolymer (A) was initially taken. First in each case an aqueous dispersion of polymer particles (B) and then in each case a thickener (C) were added to the initially taken aqueous dispersion with thorough stirring. After the homogenization of the resulting dispersion for 20 minutes, the pH was adjusted to 7 to 8 with a 25% strength ammonia solution. Thereafter, the Brookfield viscosity (spindle No. 3; 100 rpm) was determined at room temperature and the stability of the dispersion was determined in a standing cylinder with a height of fill of 100 cm.

The material composition of the aqueous polymer dispersions 1 to 8, their pH, their Brookfield viscosity and their stability are shown in the table.

TABLE
Material composition, pH, Brookfield viscosity
and stability of the aqueous dispersions 1 to 8

| | Material composition | | | | | |
|---|---|---|---|---|---|---|
| | (A) | (B) | (C) | | Viscosity | Stability |
| Ex. No. | (kg) | (kg) | (g) | pH | (mPa · s) | (months) |
| 1 | Acronal ® | Dispersion 2 | Sterocoll ® | 7.9 | 800 | 6 |
| | S 728^{a)} | Prep. Ex. 2^{d)} | HT^{f)} | | | |
| | (1.6) | (0.103) | (2.27) | | | |
| 2 | Acronal ® | Dispersion 1 | Sterocoll ® | 8 | 574 | 12 |
| | S 728^{a)} | Prep. Ex. 1^{c)} | HT^{f)} | | | |
| | (1.6) | (0.103) | (0.75) | | | |
| 3 | Acronal ® | Dispersion 2 | Sterocoll ® | 7.9 | 635 | 12 |
| | S 728^{a)} | Prep. Ex. 2^{d)} | BL^{g)} | | | |
| | (1.6) | (0.103) | (0.75) | | | |
| 4 | Acronal ® | Dispersion 1 | Xanthan | 7 | 755 | 6 |
| | S 728^{a)} | Prep. Ex. 1^{c)} | (4) | | | |
| | (1.6) | (0.103) | | | | |
| 6 | Acronal ® | Dispersion 1 | Sterocoll ® | 7 | 645 | 3 |
| | S 160^{b)} | Prep. Ex. 1^{c)} | D^{h)} | | | |
| | (1.6) | (0.103) | (0.75) | | | |
| 7 | Acronal ® | Dispersion 3 | Viskalex ® | 7 | 715 | 2 |
| | S 160^{b)} | Prep. Ex. 3^{e)} | HV 30ⁱ⁾ | | | |
| | (1.6) | (0.103) | (0.75) | | | |
| 8 | Styronal ® | Dispersion 2 | Sterocoll ® | 7 | 644 | 6 |
| | D 808^{b)} | Prep. Ex. 2^{d)} | BL^{g)} | | | |
| | (1.6) | (0.103) | (0.75) | | | |

^{a),b)}Dispersions of acrylate copolymers from BASF AG;
^{c)}Dispersion 1 of preparation example 1;
^{d)}Dispersion 2 of preparation example 2;
^{e)}Dispersion 3 of preparation example 3;
^{f),g),h)}thickener without surfactant character from BASF AG, based on copolymers of acrylic acid and/or methacrylic acid with at least one further ethylenically unsaturated monomer;
ⁱ⁾thickener without surfactant character from Ciba Specialty Chemicals, based on copolymers of acrylic acid and/or methacrylic acid with at least one further ethylenically unsaturated monomer.

The results of the table show that the polymer dispersions 1 to 8 of examples 1 to 8 have outstanding stability. Surprisingly, stabilities of up to 12 months could be achieved. The polymer dispersions 1 to 8 could thus be stored for a very long time up to their use and could be transported to different production facilities where they could be used for the production of a very wide range of marked functional products, such as, for example, marking materials, coating materials, adhesives, sealing compounds, printing inks, paper coating slips, paper sizes, starting materials for the production of shaped articles and films, catalysts, formulations of crop protection agents and cosmetic and pharmaceutical formulations. The polymer dispersions 1 to 8 proved to be extremely stable to shearing and could be circulated by pumping without problems in the circular pipelines of the production plants. The subsequent products produced from the marking materials, coating materials, adhesives, sealing compounds, printing inks, paper coating slips, paper sizes, starting materials for the production of shaped articles and films, catalysts, formulations of crop protection agents and cosmetic and pharmaceutical formulations, such as, for example, marked fuels, coatings, adhesive layers, seals, printed substrates, converted paper products, films, shaped articles, catalysts, crop protection agents, cosmetics and drugs, had outstanding performance characteristics.

### Examples 9 to 13

The preparation of the pigment-free, aqueous polymer dispersions 9 to 13 marked with fluorescent dyes and comprising the polymer particles (B4) to (B8) and a thickener (C)

Example 3 was repeated, except that, instead of the dispersion 2 of preparation example 2 comprising the polymer particles (B2), in
example 9 the dispersion 4 of preparation example 4 comprising the polymer particles (B4),
example 10 the dispersion 5 of preparation example 5 comprising the polymer particles (B5),
example 11 the dispersion 6 of preparation example 6 comprising the polymer particles (B6),
example 12 the dispersion 7 of preparation example 7 comprising the polymer particles (B7) and
example 13 the dispersion 8 of preparation example 8 comprising the polymer particles (B8)
   were used. The polymer dispersions 9 to 13 which had Brookfield viscosities of from 600 to 800 mPa·s were obtained. The polymer dispersions 9 to 13 were likewise stable for several months and outstandingly suitable for the intended uses described in examples 1 to 8.

1. A pigment-free, aqueous polymer dispersion marked with a fluorescent dye, having a viscosity of from 500 to 900 mPa·s, determined with the aid of the Brookfield method at 23° C., and comprising, based on the solid content of the polymer dispersion,
(A) from 1 to 99% by weight of at least one type of water-soluble and/or water-dispersible polymer (A),
(B) from 1 to 20% by weight of at least one type of water-dispersible polymer particle (B) which comprises at least one fluorescent dye and
(C) from 0.001 to 3% by weight of at least one thickener.

2. The polymer dispersion according to claim 1, wherein the polymer (A) is present in the form of a plurality of dispersed polymer particles (A).

3. The polymer dispersion according to claim 2, wherein the individual particle size of the dispersed polymer particles (A) are smaller than the particle size of the polymer particle (B).

4. The polymer dispersion according to claim 2, wherein the polymer particles (A) have a volume-average particle size of from 0.01 to 1 μm, determined by light scattering.

5. The polymer dispersion according to claim 1, wherein the polymer (A) is a physically drying, alternating and block, linear, branched or comb-like (co)polymer of ethylenically unsaturated monomers, or mixtures thereof which is heat-curable and curable with actinic radiation, polyaddition resins and polycondensation resins.

6. The polymer dispersion according to claim 5, wherein the polymer (A) is a copolymer of ethylenically unsaturated monomers.

7. The polymer dispersion according to claim 1, wherein the polymer particle (B) has a volume-average particle size of from 1 to 100 μm, determined by light scattering.

8. The polymer dispersion according to claim 1, wherein the fluorescent dye is dissolved in the form of a molecular dispersion in the matrix of the polymer particle (B).

9. The polymer dispersion according to claim 1, wherein the polymer particle (B) comprises from 0.001 to 10% by weight of the fluorescent dye, based on the total amount of the polymer particle (B) and the fluorescent dye.

10. The polymer dispersion according to claim 1, wherein the polymer particle (B) is obtained by a process comprising free radical suspension polymerization of ethylenically unsaturated monomers in an oil-in-water emulsion, said dispersion comprising at least one fluorescent dye in solution and having a mean particle size of at least 1 μm, determined by light scattering.

11. The polymer dispersion according to claim 10, wherein the free radical suspension polymerization is carried out in the presence of at least one surface-active compound.

12. The polymer dispersion according to claim 10, wherein the free radical suspension polymerization is carried out in the presence of at least 0.5% by weight, based on the ethylenically unsaturated monomers, of at least one hydrophobic substance.

13. The polymer dispersion according to claim 12, wherein the hydrophobic substance has a solubility in water of <0.01 g/l at 25° C. and 1013 hectopascal.

14. The polymer dispersion according to claim 10, wherein the free radical suspension polymerization is carried out in the presence of at least one protective colloid.

15. The polymer dispersion according to claim 10, wherein the free radical suspension polymerization is carried out in the presence of at least one hydrophobic ethylenically unsaturated monomer.

16. The polymer dispersion according to claim 15, wherein the free radical suspension polymerization is carried out with a mixture which comprises
at least one hydrophobic ethylenically unsaturated monomer and
at least one hydrophilic ethylenically unsaturated monomer and/or
at least one crosslinking monomer having at least two ethylenically unsaturated double bonds.

17. The polymer dispersion according to claim 1, wherein the fluorescent dye has a higher solubility in the oil phase of the oil-in-water emulsion and in the polymer particle (B) than in the aqueous phase.

18. The polymer dispersion according to claim 1, wherein the thickener is at least one selected from the group consisting of pyrogenic silica, phyllosilicate, a low molecular weight organic natural product, a low molecular weight synthetic product, a polymeric organic natural product, an oligomeric organic natural product, a modified natural product, a fully synthetic polymeric thickener, an oligomeric organic thickener without a surfactant character and an associative thickener having a surfactant character.

19. The polymer dispersion according to claim 18, wherein the thickener is selected from the group consisting of a fully synthetic polymeric thickener without a surfactant character, a fully synthetic oligomeric thickener without a surfactant character a polymeric organic natural product, an oligomeric organic natural product and a modified natural product.

20. The polymer dispersion according to claim 19, wherein the fully synthetic polymeric thickener and the fully synthetic oligomeric thickener without a surfactant character are copolymers of acrylic acid and/or methacrylic acid with at least one further ethylenically unsaturated monomer, and the polymeric organic natural product, the oligomeric organic natural product and the modified natural product are polysaccharides.

21. The polymer dispersion according to any claim 1, further comprising at least one stabilizer additive.

22. The polymer dispersion according to claim 21, wherein the stabilizer additive is selected from the group consisting of a composition for adjusting the pH, a light stabilizer, a UV absorber, a low-boiling organic solvent, a high-boiling organic solvent, a polymerization inhibitor, an antifoam, a hydrophobic substance, a surface-active compound and a protective colloid.

23. The polymer dispersion according to claim 1, which does not settle when stored at rest for at least 1.5 months.

24. A process for the preparation of a pigment-free, aqueous polymer dispersion marked with a fluorescent dye, according to claim 1, comprising homogeneously mixing in water the following components, based on the solid content of the polymer dispersion,
from 1 to 99% by weight of at least one type of water-soluble and/or water-dispersible polymer (A),
from 1 to 20% by weight of at least one type of water-dispersible polymer particle (B) which comprises at least one fluorescent dye and
from 0.001 to 3% by weight of at least one thickener and
optionally, at least one stabilizer additive.

25. The process according to claim 24, wherein the polymer (A) is in the form of an aqueous solution or dispersion and the polymer particle (B) is in the form of an aqueous dispersion.

26. The process according to claim 24, wherein the solution or the dispersion of the polymer (A) is initially taken and the dispersion of the polymer particle (B) is added to the initially taken solution or dispersion.

27. A marked, liquid, solid pulverulent and/or solid compact functional product comprising the pigment-free, aqueous polymer dispersion marked with fluorescent dyes, according to claim 1 comprising at least one effect-imparting additive differing from the stabilizer additive.

28. The product of claim 33, wherein the marked functional product is a marking material, a coating material, an adhesive, a sealing compound, a printing ink, a paper coating slip, a paper size, a starting material for the production of a shaped article or film, a catalyst, a formulation of crop protection agents and a cosmetic or pharmaceutical formulation.

29. The product according to claim 27 wherein the effect-imparting additive is selected from the group consisting of a crosslinking agent which is crosslinkable thermally and/or with actinic radiation; a reactive diluent which is heat-curable and/or curable with actinic radiation; a dye which differs from the fluorescent dyes and is soluble in water and/or in the polymer particle (A) and/or polymer particle (B); a slip additive; a catalyst for thermal crosslinking; a thermolabile free radical initiator; a photoinitiator; an adhesion promoter; a leveling agent; a film-forming assistant; a flameproofing agent; a corrosion inhibitor; a flow improver; a wax; a siccative; a biocide; a dulling agent; a deaerating agent; an organic pigment, an inorganic pigment, an opaque pigment, a transparent pigment; a metal powder; an organic filler, an inorganic filler, a transparent filler, an opaque filler; an organic nanoparticle, an nanoparticles nanoparticle; an active substance of crop protection agents; a pharmaceutical and a fragrance.

30. The product according to claim 29, wherein the organic pigment or inorganic pigment, or opaque pigment or transparent pigment is selected from the group consisting of a colored pigment, an effect pigment, an electrically conductive pigment and a magnetically shielding pigment.

31. A process for the production of a marked, liquid, solid pulverulent and solid compact functional product comprising at least one effect-imparting additive, wherein a pigment-free, aqueous polymer dispersion marked with a fluorescent dye, according to claim 1 is homogeneously mixed with at least one effect-imparting additive, resulting in a marked, liquid functional product or intermediate comprising at least one effect-imparting additive.

32. The process according to claim 31 for the production of a marked, solid pulverulent and solid compact functional product, comprising drying a marked, liquid intermediate comprising at least one effect-imparting additive.

33. A process for the production of a marked, liquid, solid pulverulent and solid compact functional product which comprise at least one effect-imparting additive, wherein a pigment-free, aqueous polymer dispersion prepared by the process according to claim 24 and marked with fluorescent dyes is homogeneously mixed with at least one effect-imparting additive, resulting in a marked, liquid functional product or intermediate comprising at least one effect-imparting additive.

34. The process according to claim 33 for the production of a marked, solid pulverulent and solid compact functional product, comprising drying a marked, liquid intermediate comprising at least one effect-imparting additive.
